(19) United States
(12) Patent Application Publication
(10) Pub. No.: US 2025/0144263 A1
(43) Pub. Date: May 8, 2025

(54) LIQUID DIFFUSING DEVICE AND CARTRIDGE AND FLUID COMBINER FOR USE THEREWITH

(71) Applicant: Aroma Republic Ltd., Tel-Aviv (IL)

(72) Inventors: Avner Sadot, Tel-Aviv (IL); Ori Jacobi, Tel-Aviv (IL); Eviatar Ben David, Hazav (IL); Elad Shalom, Beit Ezra (IL)

(21) Appl. No.: 18/833,225

(22) PCT Filed: Jan. 26, 2023
(86) PCT No.: PCT/IL2023/050091

§ 371 Date: Jul. 25, 2024

(30) Foreign Application Priority Data
Jan. 27, 2022 (IL) 290161

(51) Int. Cl. A61L 9/14

(52) U.S. Cl. CPC A61L 9/145; A61L 2209/111; A61L 2209/133; A61L 2209/134

(57) ABSTRACT

A liquid diffusing device comprising: at least two cartridge receiving portions, each configured for removably receiving a cartridge configured to contain a liquid to be diffused, and having a respective pressurized air inlet associated therewith and configured to be in fluid communication with a cartridge inlet when the cartridge is positioned in the cartridge receiving portion; a pumping mechanism configured to generate pressurized air; and a selection mechanism configured to selectively establish a fluid communication between the pumping mechanism and the pressurized air inlet of one of the cartridge receiving portions for the pumping mechanism to pump the pressurized air into the respective cartridge for diffusing the liquid contained therein and generating an aerosol flow.

### TECHNOLOGICAL FIELD

The presently disclosed subject matter relates, in general, to a liquid diffusing device, and more particularly to a liquid diffusing device capable of simultaneously accommodating more than one removable cartridges and selectively switching between those.

### BACKGROUND

Liquid diffusing devices have been known and been used for delivering aromatic sprays in the air. There have been devices with removable and/or re-fillable cartridges containing aromatic liquids that are diffused into the surroundings.

There exists a need for a liquid diffusing device and a removable cartridge for use therewith that is more adaptive and is spill-proof. Also, there exists a need for a liquid diffusing device that can deliver the aroma directionally.

### GENERAL DESCRIPTION

The presently disclosed subject matter is related to a liquid diffusing device for delivering aerosol flow from a liquid contained in a cartridge into an environment, a cartridge to be used therewith, and a fluid combiner to be used therewith. The cartridge comprises a liquid that is to be diffused and delivered to the environment. The environment can be a room, office space, restaurant, etc. and the liquid can be an aromatic liquid and the intended application of the liquid diffusing device to deliver fragrance to the environment. In another application, the liquid diffusing device can be used to deliver medicine or drug in the form of a spray in an environment.

According to an aspect of the presently disclosed subject matter, there is provided a liquid diffusing device comprising: at least two cartridge receiving portions, each configured for removably receiving a cartridge configured to contain a liquid to be diffused, and having a respective pressurized air inlet associated therewith and configured to be in fluid communication with a cartridge inlet when the cartridge is positioned in the cartridge receiving portion; a pumping mechanism configured to generate pressurized air; and a selection mechanism configured to selectively establish a fluid communication between the pumping mechanism and the pressurized air inlet of one of the cartridge receiving portions for the pumping mechanism to pump the pressurized air into the respective cartridge for diffusing the liquid contained therein and generating an aerosol flow.

It is to be understood herein that for the purposes of the present description, the cartridge receiving portion can be considered as an portion of the liquid diffusing device that engages and/or contributes to holding the cartridge when the cartridge is introduced and positioned within the liquid diffusing device. In different examples of the first aspect, the liquid diffusing device can have any number (more than two) of cartridge receiving portions that can be selected and shuffled among themselves by the selection mechanism.

Any such cartridge receiving portion whose associated pressurized air inlet is in fluid communication with the pumping mechanism is to be considered as the selected cartridge receiving portion at that time for the purpose of understanding of the present description.

The pumping mechanism can comprise a pump outlet, and the selection mechanism can be configured to selectively align the pressurized air inlet of said one of the cartridge receiving portions with said pump outlet to establish said fluid communication therebetween.

In some examples, the pumping mechanism can be in fluid communication with each of the cartridge receiving portions and the selection mechanism can be configured to selectively open and close those fluid communications. For instance, the pump mechanism can be in fluid communication via respective conduits to each of the cartridge receiving portions and the selection mechanism can be configured to selectively block and unblock one or more of those pipes. In such an example, more than one cartridge receiving portion can be the selected cartridge receiving portion at a time.

The pump outlet can comprise an outlet sealing member configured to seal the fluid communication between the pressurized air inlet and the pump outlet.

The at least two cartridge receiving portions can constitute at least a part of a movable assembly and said pumping mechanism constitutes at least a part of a stationary assembly. The selection mechanism can be configured to move the movable assembly with respect to the stationary assembly. In some examples, the selection mechanism can comprise a motor configured to rotate the movable assembly about a rotational axis with respect to the stationary assembly. In such examples, the movable assembly can be a cartridge receiving tray including the cartridge receiving portions, and forming a carousel like arrangement where the cartridge receiving portions are rotatable about a longitudinal axis of the liquid diffusing device, which constitutes the rotational axis. The selection mechanism can further include a gear arrangement to rotate the carousel-like cartridge receiving tray. The liquid diffusing device can further comprise a mounting mechanism configured to movably mount said movable assembly to said stationary assembly. The stationary assembly can include a base member to which the cartridge receiving tray can be mounted and can be movable with respect thereto.

In some examples, the liquid diffusing device can comprise a central air conduit constituting a part of the stationary assembly and the movable assembly is configured to rotate around said central air conduit. The central air conduit can be coaxial with the longitudinal axis.

The outlet sealing member of the pump outlet can seal the connection between the pump outlet and the pressurized air inlet and simultaneously allows the pressurized air inlet to slide over the pump outlet when the selection mechanism moves the cartridge receiving portion.

The liquid diffusing device can have a controller that is configured to control the operation of the selection mechanism for selecting the cartridge receiving portions and aligning the selected cartridge receiving portion such the corresponding pressurized air inlet is aligned with and in fluid communication with the pump outlet. The controller can control the operation of the motor to rotate the cartridge receiving tray with respect to the base member to cause the alignment and establishment of the fluid communication. The controller can be configured to operate according to the instructions fed to the controller in any of the manners and to follow a predetermined pattern that may define a duration of operation for each one of the cartridges, the pressure of the pumping mechanism for each one of the cartridges, etc.

The controller can be configured to determine whether a cartridge receiving portion has a cartridge or not and can be configured to operate the pumping mechanism only if the selected cartridge receiving portion includes a cartridge. The controller can be configured to control the operation of the pumping mechanism in coordination with that of the selection mechanism. For instance, the controller switches on the pumping mechanism only after the selected cartridge receiving portion is in fluid communication with the pumping mechanism. In other words, only after having controlled the selection mechanism to select one of the cartridge receiving portions and establish a fluid communication thereof with the pumping mechanism does the controller switches on the pumping mechanism to pump the pressurized air into the corresponding cartridge. Once a next cartridge receiving portion is to be selected, the controller switches off the pumping mechanism prior to controlling the selection mechanism to move the cartridge receiving tray. In other words, the controller switches off the pumping mechanism prior to terminating the fluid communication between the previously selected cartridge receiving portion and the pumping mechanism.

In some examples, the liquid diffusing device can further comprise a blower, which can constitute a part of the stationary assembly, and is configured to generate a blown air in a blowing direction. The cartridge receiving portions are so aligned that when the selected cartridge receiving portion is in fluid communication with the pumping mechanism, a cartridge outlet of the corresponding cartridge is so positioned that the aerosol flow being discharged therefrom is aligned with the blown air generated by the blower. Thus, the device so operates that the pumping mechanism pumps the pressurized air into the cartridge causing the liquid contained therein to be diffused into the aerosol flow. The aerosol is then discharged from the cartridge outlet which is aligned with the blown air, that interacts with and directs the aerosol flow in the blowing direction into the atmosphere around the device.

The central air conduit can be in fluid communication with the blower and can have a blown air outlet configured to discharge therefrom the blown air. The central air conduit can have a conduit body extending along the longitudinal axis.

The controller can be configured to operate the blower in coordination with the selection mechanism. For instance, the controller switches on the blower only after the selected cartridge receiving portion is aligned with the blown air. In other words, only after having controlled the selection mechanism to select one of the cartridge receiving portions and align the same such that the corresponding cartridge outlet will be aligned with the blown air, does the controller switches on the blower to direct the aerosol flow in the blowing direction. Once a next cartridge receiving portion is to be selected, the controller switches off the blower prior to controlling the selection mechanism to move the cartridge receiving tray thereby de-aligning the cartridge receiving portion from the blown air.

In fact, the controller can be configured to control the operation of the pumping mechanism, the selection mechanism, and the blower in coordination with each other, i.e., to follow a sequence. First of all, the selection mechanism is controlled to select a cartridge receiving portion, establish the fluid communication thereof with the pumping mechanism, and simultaneously position the cartridge outlet so as to be aligned with the blown air. Then, the blower is switched on. After that, the pumping mechanism is switched on in order to pump the pressurized air and generate the aerosol flow. The blower is switched on prior to the pumping mechanism so that the blown air is already generated to blow the aerosol flow and prevent any wastage of the aerosol. Once a next cartridge receiving portion is to be selected, first of all the pumping mechanism is switched off to stop the generation of the aerosol flow. Then, the blower is switched off. The blower is switched off after the pumping mechanism so that the blown air blows away the already generated aerosol and little or no aerosol is wasted. After that the selection mechanism is controlled to move the cartridge receiving tray to select the next one of the cartridge receiving portions.

The controller is configured for controlling the operation of the blower according to a predetermined pattern that may define a duration of operation of the blower and/or speed of the blower for each one of the cartridges, etc. In other words, the controller can be configured to operate the liquid diffusing device according to a predetermined pattern that may define a duration of operation for each one of the cartridges, the pressure of the pumping mechanism for each one of the cartridges, a duration of operation of the blower and/or speed of the blower for each one of the cartridges, etc.

In some examples, the controller can be fed instructions for accordingly operating the liquid diffusing device. The device can have a reader module that can read a corresponding code positioned on the cartridge to receive instructions for the controller for operating the selection mechanism, pumping mechanism, and the blower accordingly with respect to that cartridge. In some examples, the device can be configured to receive instructions from and transmit data to a user device remotely through a wireless connection. In some examples, the device can include a controller readable memory and the instructions can be stored on the memory.

According to a second aspect of the presently disclosed subject matter, there is provided a liquid diffusing device comprising: at least one cartridge receiving portion configured for removably receiving a cartridge configured to contain a liquid to be diffused; a pumping mechanism configured to generate pressurized air, and to be in fluid communication with the cartridge for pumping the pressurized air into the cartridge for diffusing the liquid contained therein and generating an aerosol flow; a blower configured to generate a blown air in a blowing direction, said blown air being configured to direct said aerosol flow in the blowing direction away from the liquid diffusing device; and a controller configured to control the operation of the pumping mechanism and the blower in a predetermined coordinated manner.

The controller can be configured to provide said coordination by switching on the blower prior to switching on the pumping mechanism. The controller can be configured to provide said coordination by switching off the pumping mechanism prior to switching off the blower. For instance, upon receiving a signal to operate the device, the controller can be configured to switch on the blower prior to switching on the pumping mechanism, and upon receiving a signal to turn off the device, the controller can be configured to switch off the pumping mechanism prior to switching off the blower.

According to a third aspect of the presently disclosed subject matter, there is provided a cartridge for use with a liquid diffusing device, said cartridge comprising: a reservoir containing a liquid to be diffused; a cartridge outlet in fluid communication with the reservoir; a locking mechanism configured to displace between a closed state at which the cartridge outlet is sealed and an open state at which the cartridge outlet is open, the locking mechanism, at its closed state, being configured to prevent the introduction of the cartridge in the liquid diffusing device.

The cartridge can be used with a liquid diffusing device, for example the liquid diffusing devices of first and second aspects, for delivering aromatic spray into an environment. The cartridge can be configured to receive pressurized air through a cartridge inlet for diffusing the liquid contained therein into an aerosol flow by action of the pressurized air. The aerosol flow is then delivered through the cartridge outlet.

The cartridge can have a cartridge body and the locking mechanism can have an obstructing member that protrudes from the cartridge body in the closed state of the locking mechanism. For instance, the obstructing member can have an obstructing portion that protrudes further from a contour defined by the cartridge body and would engage with the cartridge receiving portion of the liquid diffusing device, for example a rim R thereof. It is to be understood herein that the liquid diffusing device can be any liquid diffusing device having dimensions of the cartridge receiving portions corresponding to those of the cartridge with its locking mechanism in open state, and with the obstructing member extending beyond those dimensions in the closed state to engage any portion of the liquid diffing device for preventing the introduction of the cartridge.

The cartridge body can have a body side wall and the obstructing member, in fact the obstructing portion, protrudes radially from the body side wall in the closed state of the locking mechanism. The body side wall can have a shape and size corresponding the shape and size of the cartridge receiving portion and the obstructing portion extending radially from the body side wall can engage the rim of the cartridge receiving portion thereby preventing the introduction of the cartridge into the cartridge receiving portion. In the open state of the locking mechanism, the obstructing member is displaced radially inwardly and the obstructing portion lies within the contour defined by the body side wall. In such a state, the cartridge can be inserted into the cartridge receiving portion.

The locking mechanism can constitute a part of a cover of the cartridge. The obstructing member can have an orifice that corresponds in shape and size to the cartridge outlet and in the open state of the locking mechanism, the orifice aligns with the cartridge outlet thereby opening the outlet for the aerosol flow to be discharged therefrom. In the locked state of the locking mechanism, the orifice can be not aligned with the cartridge outlet and the obstructing member can seal the outlet. Thus, the locking mechanism simultaneously allows introduction of the cartridge into the corresponding cartridge receiving portion and opens the cartridge outlet for use thereof. In some examples, the locking mechanism can have a biasing member configured to bias the obstructing member to keep the locking mechanism into its closed state, such that as soon as the cartridge is removed from the cartridge receiving portion, the biasing member displaces the locking mechanism into its closed state thereby sealing the cartridge outlet. This prevents spillage of the liquid from the cartridge through the cartridge outlet. In some examples, the cartridge can include a one-way valve at the cartridge inlet configured to allow the pressurized air to enter the cartridge while simultaneously preventing the spillage of the liquid from the cartridge inlet. In some examples, the cartridge can comprise a venturi device configured for diffusing the liquid contained in the cartridge by the action of the pressurized air.

According to a fourth aspect of the presently disclosed subject matter, there is provided a fluid combiner configured for use with a liquid diffusing device having a blower and a pumping mechanism, said liquid diffusing device further having a cartridge comprising a cartridge outlet and configured to discharge an aerosol flow therefrom, said combiner comprising: a combiner body with a fluid channel; a first combiner inlet formed in the combiner body, and configured to be in fluid communication with the blower to receive blown air therefrom into the fluid channel; a second combiner inlet formed in the combiner body, and configured to be in fluid communication with the cartridge outlet to receive the aerosol flow into the fluid channel; a combiner outlet in fluid communication with the fluid channel for discharging a mixture of the blown air and the aerosol flow therefrom.

The fluid channel can constitute a fluid path extending from the first combiner inlet to the combiner outlet. The second combiner inlet is positioned between the first combiner inlet and the combiner outlet. The second combiner inlet can be closer to the first combiner inlet than to the combiner outlet. This prevents a direct access by fingers into the cartridge, or prevents the dust to enter into the cartridge. Apart from that, this positioning of the second combiner inlet allows a longer distance to the aerosol flow to travel within the fluid channel thereby allowing more distance and time for the big droplets in the aerosol to settle down in the fluid channel in flow back into the cartridge. The fluid channel can comprise a channel internal surface facing an interior of the fluid channel and having a first surface portion extending between the first combiner inlet and the second combiner inlet, and a second surface portion extending between the combiner outlet and the second combiner inlet, wherein said first and second surface portions are sloped down into the second combiner inlet. The fluid channel can be configured to permit the blown air to direct the aerosol flow out of the combiner outlet. The fluid combiner can constitute at least a part of a cover for the cartridge.

The liquid diffusing device according to the first and second aspects and/or the cartridge according to the third aspect can include the fluid combiner according to the fourth aspect of the presently disclosed subject matter. The liquid diffusing device can include a fluid combiner corresponding to and associated with each one of the cartridge receiving portions. Alternatively, there can be only one fluid combiner constituting a part of the stationary assembly and the selected cartridge receiving portion can be aligned with that one fluid combiner in the manner similar as the cartridge receiving portion is aligned with the pump outlet.

In the examples, where the liquid diffusing device has the fluid combiner and the central air conduit along with the blower, the first combiner inlet of the fluid combiner corresponding to the selected cartridge receiving portion is aligned with and in fluid communication with the blown air outlet of the central air conduit. At the same time, the conduit body is so aligned with the first combiner inlets of the other fluid combiners (corresponding to the non-selected cartridge receiving portions) and so as to prevent access by fingers into those fluid combiners and corresponding cartridges. In some examples, the liquid diffusing device can have a conduit cover positioned on top of the conduit body such that the conduit cover prevents access into first combiner inlets of the fluid combiners corresponding to the non-selected cartridge receiving portions.

It is to be understood herein that in some examples, the liquid diffusing device may not include the blower and in such examples, the central air conduit may also not be present and the liquid diffusing device can have an empty central path instead or that central path can accommodate any other components of the liquid diffusing device. Also, in such examples, the liquid diffusing device may not include the fluid combiners.

In some examples, the liquid diffusing device can include the fluid combiner with only one cartridge receiving portion and without any selection mechanism. For instance, such a liquid diffusing device can have a cartridge (removable or fixed) to generate an aerosol flow, a blower to direct the aerosol flow, and a fluid combiner similar to the one described above to facilitate the above described interaction between the aerosol flow and blown air from the blower.

In the examples, where the cartridge includes the fluid combiner, the fluid combiner can comprise combiner connection elements configured to be connected to the cartridge. The cartridge cover can have corresponding cartridge connection elements for connecting to the combiner connection elements. The combiner connection elements and the cartridge connection elements can constitute a quick-fitting connection arrangement such as a snap connection. In other examples, the connection arrangement can be any other quick-connection arrangement know to a skilled person.

The locking mechanism can be positioned between the fluid combiner and the cartridge when the fluid combiner is connected to the cartridge. In some examples, the cartridge can include the fluid combiner without the locking mechanism and can operate in the same manner as described above. For instance, such a cartridge can be used with a liquid diffusing device including a blower to direct the aerosol flow, and can include a fluid combiner similar to the one described above to facilitate the above described interaction between the aerosol flow and blown air from the blower.

According to a fifth aspect of the presently disclosed subject matter, there is provided a liquid diffusing device comprising the fluid combiner according to the fourth aspect of the presently disclosed subject matter.

According to a sixth aspect of the presently disclosed subject matter, there is provided a cartridge for use with a liquid diffusing device, said cartridge comprising a fluid combiner, said combiner comprising:
a combiner body with a fluid channel;
a first combiner inlet formed in the combiner body, and configured to be in fluid communication with a blower of the liquid diffusing device to receive the blown air therefrom into the fluid channel;
a second combiner inlet formed in the combiner body, and configured to be in fluid communication with the cartridge outlet to receive the aerosol flow into the fluid channel;
a combiner outlet in fluid communication with the fluid channel for discharging the aerosol flow having directed by the blown air.

### EMBODIMENTS

A more specific description is provided in the Detailed Description whilst the following are non-limiting examples of different embodiments of the presently disclosed subject matter. It should be appreciated that Embodiments 1 to 35, correspond to the first aspect of the presently disclosed subject matter; Embodiments 36 to 38, correspond to the second aspect of the presently disclosed subject matter; Embodiments 39 to 52, correspond to the third aspect of the presently disclosed subject matter; Embodiments 53 to 59, correspond to the fourth aspect of the presently disclosed subject matter; Embodiment 60, corresponds to the fifth aspect of the presently disclosed subject matter; and Embodiment 61, correspond to the sixth aspect of the presently disclosed subject matter;
1. A liquid diffusing device comprising:
at least two cartridge receiving portions, each configured for removably receiving a cartridge configured to contain a liquid to be diffused, and having a respective pressurized air inlet associated therewith and configured to be in fluid communication with a cartridge inlet when the cartridge is positioned in the cartridge receiving portion;
a pumping mechanism configured to generate pressurized air; and
a selection mechanism configured to selectively establish a fluid communication between the pumping mechanism and the pressurized air inlet of one of the cartridge receiving portions for the pumping mechanism to pump the pressurized air into the respective cartridge for diffusing the liquid contained therein and generating an aerosol flow.
2. The liquid diffusing device according to Embodiment 1, wherein the pumping mechanism comprises a pump outlet, and the selection mechanism is configured to selectively align the pressurized air inlet of said one of the cartridge receiving portions with said pump outlet to establish said fluid communication therebetween.
3. The liquid diffusing device according to Embodiment 2, wherein said pump outlet comprises an outlet sealing member configured to seal the fluid communication between the pressurized air inlet and the pump outlet.
4. The liquid diffusing device according to any one of Embodiments 1 to 3, wherein said at least two cartridge receiving portions constitute at least a part of a movable assembly, and said pumping mechanism constitutes at least a part of a stationary assembly.
5. The liquid diffusing device according to Embodiment 4, wherein said selection mechanism is configured to move the movable assembly with respect to the stationary assembly.
6. The liquid diffusing device according to Embodiment 4 or 5, wherein said selection mechanism comprises a motor configured to rotate the movable assembly about a rotational axis with respect to the stationary assembly.
7. The liquid diffusing device according to Embodiment 6, wherein the liquid diffusing device comprises a central air conduit constituting a part of the stationary assembly and the movable assembly is configured to rotate around said central air conduit.
8. The liquid diffusing device according to any one of Embodiments 4 to 7, further comprising a mounting mechanism configured to movably mount said movable assembly to said stationary assembly.
9. The liquid diffusing device according to any one of Embodiments 1 to 8, further comprising a controller configured for controlling the operation of the selection mechanism to select the cartridge receiving portions to establish the fluid communication between the selected cartridge receiving portion with the pumping mechanism.
10. The liquid diffusing device according to Embodiment 9, wherein said controller is configured to determine whether a cartridge receiving portion has a cartridge or not.
11. The liquid diffusing device according to Embodiment 9 or 10, wherein said controller is configured to control the operation of the pumping mechanism in coordination with that of the selection mechanism.
12. The liquid diffusing device according to Embodiment 11, wherein the controller is configured to provide said coordination by switching on the pumping mechanism after said selection mechanism has established the fluid communication between at least one of the cartridge receiving portions and the pumping mechanism.
13. The liquid diffusing device according to any one of Embodiments 9 to 11, wherein the controller is configured to provide said coordination by switching off the pumping mechanism prior to said selection mechanism terminating the fluid communication between said at least one of the cartridge receiving portions and the pumping mechanism.
14. The liquid diffusing device according to any one of Embodiments 9 to 13, wherein the controller is configured for controlling the operation of the pumping mechanism according to a predetermined pattern.
15. The liquid diffusing device according to any one of Embodiments 1 to 14, further comprising a blower configured to generate a blown air directed towards a blowing direction.
16. The liquid diffusing device according to Embodiment 15, wherein the selection mechanism is configured to selectively align said one of the cartridge receiving portions with the blown air and thereby causing the aerosol flow to be directed by the blown air towards the blowing direction.
17. The liquid diffusing device according to Embodiment 15 or 16, when dependent on claim 4, wherein said blower constitutes at least a part of the stationary assembly.
18. The liquid diffusing device according to any one of Embodiments 15 to 17, when dependent on claim 9, wherein said controller is configured to control the operation of the blower in coordination with that of the selection mechanism.
19. The liquid diffusing device according to Embodiment 18, wherein the controller is configured to provide said coordination by switching on the blower after said selection mechanism has aligned said one of the cartridge receiving portions with the blown air.
20. The liquid diffusing device according to Embodiment 18 or 19, wherein the controller is configured to provide said coordination by switching off the blower prior to said selection mechanism de-aligning said one of the cartridge receiving portions from the blown air.
21. The liquid diffusing device according to any one of Embodiments 18 to 20, wherein the controller is configured for controlling the operation of the blower according to a predetermined pattern.
22. The liquid diffusing device according to any one of Embodiments 15 to 21, when dependent on claim 11, wherein said controller is configured to control the operation of the blower in coordination with that of the pumping mechanism.
23. The liquid diffusing device according to Embodiment 22, wherein the controller is configured to provide said coordination between the blower and the pumping mechanism by switching on the blower prior to switching on the pumping mechanism.
24. The liquid diffusing device according to Embodiment 22 or 23, wherein the controller is configured to provide said coordination between the blower and the pumping mechanism by switching off the blower after switching off the pumping mechanism.
25. The liquid diffusing device according to any one of Embodiments 15 to 24, further comprising a fluid combiner, said combiner comprising:
a combiner body with a fluid channel;
a first combiner inlet formed in the combiner body, and configured to be in fluid communication with the blower to receive the blown air therefrom into the fluid channel;
a second combiner inlet formed in the combiner body, and configured to be in fluid communication with a cartridge outlet of a cartridge to receive the aerosol flow into the fluid channel;
a combiner outlet in fluid communication with the fluid channel for discharging the aerosol flow having directed by the blown air in the blowing direction.
26. The liquid diffusing device according to Embodiment 25, wherein the fluid channel constitutes a fluid path extending from the first combiner inlet to the combiner outlet.
27. The liquid diffusing device according to Embodiment 26, wherein the second combiner inlet is positioned between the first combiner inlet and the combiner outlet.
28. The liquid diffusing device according to Embodiment 27, wherein the second combiner inlet is closer to the first combiner inlet than to the combiner outlet.
29. The liquid diffusing device according to any one of Embodiments 25 to 28, wherein the fluid channel comprises a channel internal surface facing an interior of the fluid channel and having a first surface portion extending between the first combiner inlet and the second combiner inlet, and a second surface portion extending between the combiner outlet and the second combiner inlet, wherein said first and second surface portions are sloped down into the second combiner inlet.
30. The liquid diffusing device according to any one of Embodiments 25 to 29, wherein the fluid combiner is associated with at least one of the cartridge receiving portions, wherein the selection mechanism is configured to align said first combiner inlet with the blown air simultaneously with establishing the fluid communication between the pumping mechanism and said one of the cartridge receiving portions.
31. The liquid diffusing device according to any one of Embodiments 25 to 30, wherein said fluid combiner constitutes at least a part of a cover for the cartridge at least when the cartridge is received in the corresponding cartridge receiving portion.
32. The liquid diffusing device according to Embodiment 31, wherein the fluid combiner is snap connectable to the cartridge.
33. The liquid diffusing device according to any one of Embodiments 25 to 32, when dependent on claim 7, wherein the central air conduit is in fluid communication with the blower and has a blown air outlet configured to discharge said blown air.
34. The liquid diffusing device according to Embodiment 33, wherein the liquid diffusing device comprises at least two fluid combiners, each associated with one the cartridge receiving portions, wherein the selection mechanism is configured to align said first combiner inlet of one of the fluid combiners with the blown air outlet simultaneously with establishing the fluid communication between the pumping mechanism and corresponding one of the cartridge receiving portions.
35. The liquid diffusing device according to Embodiment 34, wherein the central air conduit blocks the first combiner inlets of the fluid combiners corresponding to the remaining cartridge receiving portions.
36. A liquid diffusing device comprising:
at least one cartridge receiving portion configured for removably receiving a cartridge configured to contain a liquid to be diffused;
a pumping mechanism configured to generate pressurized air, and to be in fluid communication with the cartridge for pumping the pressurized air into the cartridge for diffusing the liquid contained therein and generating an aerosol flow;
a blower configured to generate a blown air in a blowing direction, said blown air being configured to direct said aerosol flow in the blowing direction away from the liquid diffusing device; and
a controller configured to control the operation of the pumping mechanism and the blower in a predetermined coordinated manner.
37. The liquid diffusing device according to Embodiment 36, wherein the controller is configured to provide said coordination by switching on the blower prior to switching on the pumping mechanism.
38. The liquid diffusing device according to Embodiment 36 or 37, wherein the controller is configured to provide said coordination by switching off the pumping mechanism prior to switching off the blower.
39. A cartridge for use with a liquid diffusing device, said cartridge comprising;
a reservoir containing a liquid to be diffused;
a cartridge outlet in fluid communication with the reservoir;
a locking mechanism configured to displace between a closed state at which the cartridge outlet is sealed and an open state at which the cartridge outlet is open, the locking mechanism, at its closed state, being configured to prevent the introduction of the cartridge in the liquid diffusing device.
40. The cartridge according to Embodiment 39, wherein the cartridge comprises a cartridge body, and the locking mechanism comprises an obstructing member configured to protrude from the cartridge body in the closed state of the locking mechanism.
41. The cartridge according to Embodiment 40, wherein the obstructing member, in the closed state of the locking mechanism, is configured to engage at least a part of the liquid diffusing device during introduction of the cartridge in the liquid diffusing device, thereby preventing said introduction of the cartridge.
42. The cartridge according to Embodiment 40 or 41, wherein the cartridge body comprises a body side wall and the obstructing member is configured to protrude radially from the body side wall in the closed state of the locking mechanism.
43. The cartridge according to any one of Embodiments 39 to 42, wherein the locking mechanism constitutes a part of a cover of the cartridge.
44. The cartridge according to any one of Embodiments 39 to 43, comprises a venturi device.
45. The cartridge according to any one of Embodiments 39 to 44, further comprising a fluid combiner, said combiner comprising:
a combiner body with a fluid channel;
a first combiner inlet formed in the combiner body, and configured to be in fluid communication with a blower of the liquid diffusing device to receive the blown air therefrom into the fluid channel;
a second combiner inlet formed in the combiner body, and configured to be in fluid communication with the cartridge outlet to receive the aerosol flow into the fluid channel;
a combiner outlet in fluid communication with the fluid channel for discharging the aerosol flow having directed by the blown air.
46. The cartridge according to Embodiment 45, wherein the fluid channel constitutes a fluid path extending from the first combiner inlet to the combiner outlet.
47. The cartridge according to Embodiment 46, wherein the second combiner inlet is positioned between the first combiner inlet and the combiner outlet.
48. The cartridge according to Embodiment 47, wherein the second combiner inlet is closer to the first combiner inlet than to the combiner outlet.
49. The cartridge according to any one of Embodiments 45 to 48, wherein the fluid channel comprises a channel internal surface facing an interior of the fluid channel and having a first surface portion extending between the first combiner inlet and the second combiner inlet, and a second surface portion extending between the combiner outlet and the second combiner inlet, wherein said first and second surface portions are sloped down into the second combiner inlet.
50. The cartridge according to any one of Embodiments 45 to 49, wherein said fluid combiner constitutes at least a part of a cover for the cartridge.
51. The cartridge according to Embodiment 50, wherein the fluid combiner is snap connectable to the cartridge.
52. The cartridge according to any one of Embodiments 45 to 51, wherein said locking mechanism is positioned between the fluid combiner and the reservoir.
53. A fluid combiner configured for use with a liquid diffusing device having a blower and a pumping mechanism, said liquid diffusing device further having a cartridge comprising a cartridge outlet and configured to discharge an aerosol flow therefrom, said combiner comprising:
a combiner body with a fluid channel;
a first combiner inlet formed in the combiner body, and configured to be in fluid communication with the blower to receive blown air therefrom into the fluid channel;
a second combiner inlet formed in the combiner body, and configured to be in fluid communication with the cartridge outlet to receive the aerosol flow into the fluid channel;
a combiner outlet in fluid communication with the fluid channel for discharging a mixture of the blown air and the aerosol flow therefrom.
54. The fluid combiner according to Embodiment 53, wherein the fluid channel constitutes a fluid path extending from the first combiner inlet to the combiner outlet.
55. The fluid combiner according to Embodiment 54, wherein the second combiner inlet is positioned between the first combiner inlet and the combiner outlet.
56. The cartridge according to Embodiment 55, wherein the second combiner inlet is closer to the first combiner inlet than to the combiner outlet.
57. The fluid combiner according to any one of Embodiments 53 to 56, wherein the fluid channel comprises a channel internal surface facing an interior of the fluid channel and having a first surface portion extending between the first combiner inlet and the second combiner inlet, and a second surface portion extending between the combiner outlet and the second combiner inlet, wherein said first and second surface portions are sloped down into the second combiner inlet.
58. The fluid combiner according to any one of Embodiments 53 to 57, wherein the fluid channel is configured to permit the blown air to direct the aerosol flow out of the combiner outlet.
59. The fluid combiner according to any one of Embodiments 53 to 58, wherein said fluid combiner constitutes at least a part of a cover for the cartridge.
60. A liquid diffusing device comprising the fluid combiner according to any one of Embodiments 53 to 59.
61. A cartridge for use with a liquid diffusing device, said cartridge comprising a fluid combiner, said combiner comprising:
a combiner body with a fluid channel;
a first combiner inlet formed in the combiner body, and configured to be in fluid communication with a blower of the liquid diffusing device to receive the blown air therefrom into the fluid channel;
a second combiner inlet formed in the combiner body, and configured to be in fluid communication with the cartridge outlet to receive the aerosol flow into the fluid channel;
a combiner outlet in fluid communication with the fluid channel for discharging the aerosol flow having directed by the blown air.

Further aspects and embodiments of the presently disclosed subject matter are described below with reference to the accompanying drawings.

### BRIEF DESCRIPTION OF THE DRAWINGS

In order to better understand the subject matter that is disclosed herein and to exemplify how it may be carried out in practice, embodiments will now be described, by way of non-limiting examples only, with reference to the accompanying drawings, in which:

FIG. 1A illustrates a perspective view of a liquid diffusing device according to one example of the presently disclosed subject matter;

FIG. 1B illustrates a cross-section taken along line A-A in FIG. 1A;

FIG. 1C illustrates the liquid diffusing device of FIG. 1A with some of its components removed for illustration purposes;

FIG. 1D illustrates a cross-section taken along line B-B in FIG. 1C;

FIG. 1E illustrates an enlarged view of section Al of FIG. 1D;

FIG. 2A illustrates a perspective view of a liquid diffusing device according to another example of the presently disclosed subject matter;

FIG. 2B illustrates a cross-section taken along line C-C in FIG. 2A;

FIG. 2C illustrates a top perspective view of the liquid diffusing device of FIG. 2A;

FIG. 2D illustrates a partial exploded view of the liquid diffusing device of FIG. 2A;

FIG. 2E illustrates a bottom perspective view of the liquid diffusing device as shown in FIG. 2D;

FIG. 2F illustrates a perspective view of the liquid diffusing device of FIG. 2A with additional components, i.e., blower and fluid combiners;

FIG. 2G illustrates a cross-section taken along line D-D in FIG. 2F;

FIG. 2H illustrates a top perspective view of the liquid diffusing device as shown in FIG. 2G;

FIG. 3A illustrates a perspective view of a cartridge with its locking mechanism in closed state according to an example of the presently disclosed subject matter;

FIG. 3B illustrates a cross-section taken along line E-E in FIG. 3A;

FIG. 3C illustrates a perspective view of the cartridge of FIG. 3A with its locking mechanism in open state;

FIG. 3D illustrates a cross-section taken along line F-F in FIG. 3C;

FIG. 3E illustrates the cartridge of FIG. 3A with its locking mechanism in closed state along with a cartridge receiving portion;

FIG. 3F illustrates the cartridge of FIG. 3C with its locking mechanism in open state along with a cartridge receiving portion;

FIG. 3G illustrates a perspective view of the cartridge of FIG. 3A with additional component, i.e., a fluid combiner;

FIG. 3H illustrates a cross-section taken along line G-G in FIG. 3G;

FIG. 3I illustrates a partial exploded view of the cartridge of FIG. 3G;

FIG. 3J illustrates a cross-section taken along line H-H in FIG. 3I;

FIG. 4A illustrates a top perspective view of a fluid combiner according to an example of the presently disclosed subject matter;

FIG. 4B illustrates a cross-section taken along line I-I in FIG. 4A; and

FIG. 4C illustrates a bottom perspective view of the fluid combiner of FIG. 4A.

### DETAILED DESCRIPTION OF EMBODIMENTS

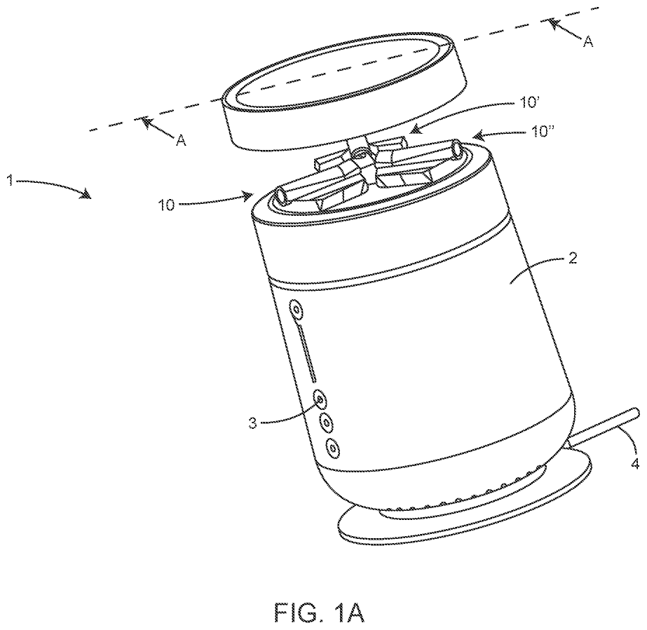

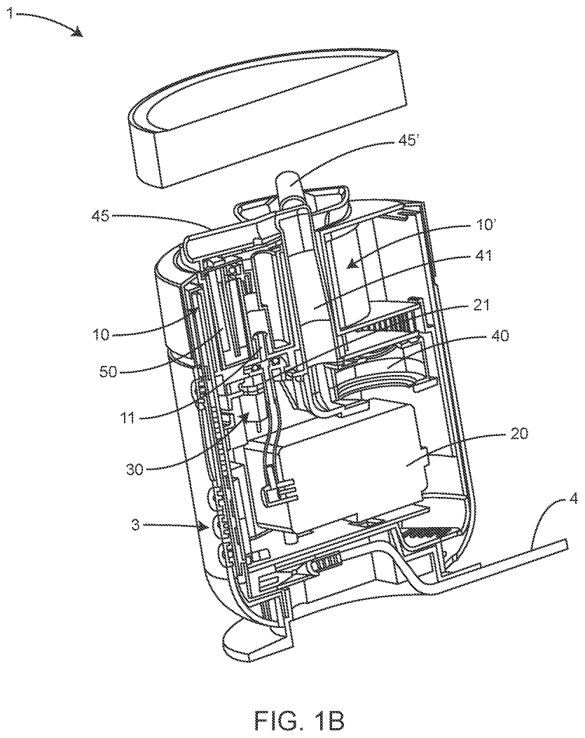

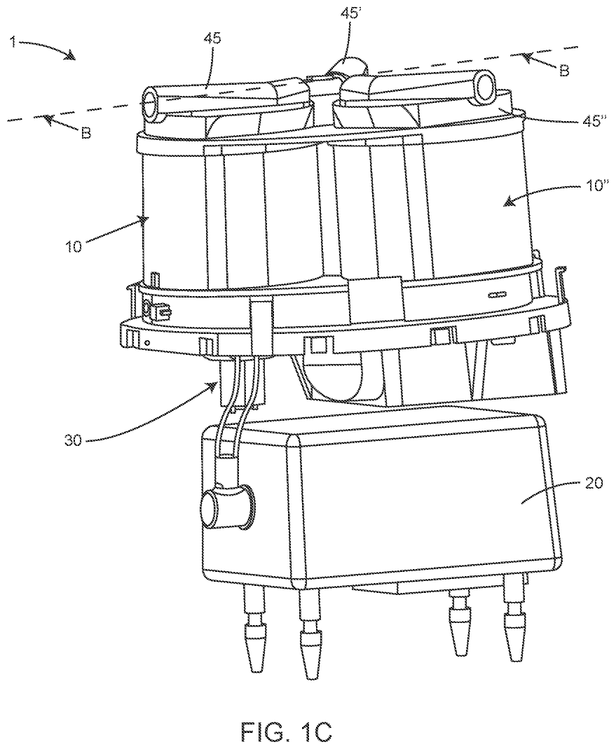

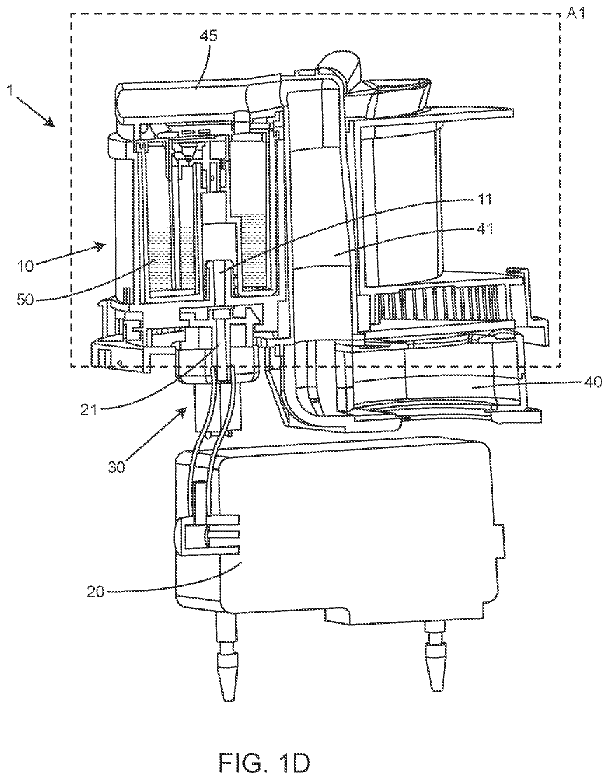

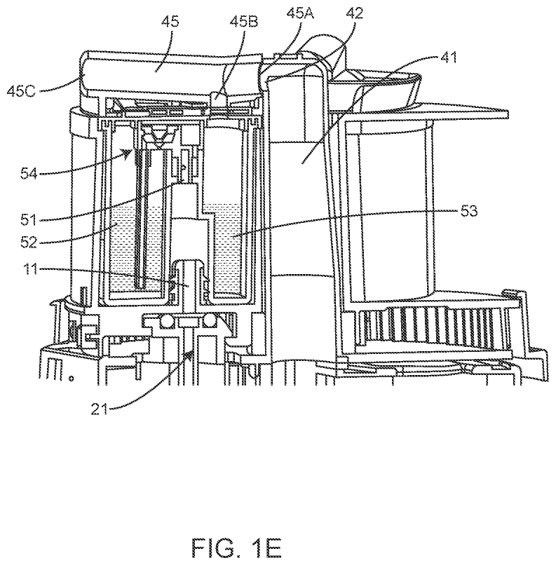

Reference is first made to FIGS. 1A to 1E to describe a liquid diffusing device 1 and its operation according to an example of the presently disclosed subject matter. The liquid diffusing device has three cartridge receiving portions 10, 10′, and 10″, each of which has a respective cartridge, out of which one cartridge 50 is visible. The liquid diffusing device 1 further has a pumping mechanism 20 configured to generate pressurized air. The operation of the liquid diffusing device 1 has been described below with respect to the cartridge receiving portion 10 and corresponding cartridge 50, and it is to be understood herein that the cartridge receiving portions and their corresponding cartridges operate similarly.

The liquid diffusing device 1 has a selection mechanism 30 configured to select one of the cartridge receiving portions at a time and establish a fluid communication between the selected cartridge receiving portion, which in the illustrated example is the cartridge receiving portion 10, and the pumping mechanism 20. The cartridge receiving portion 10 has a pressurized air inlet 11 configured to be in fluid communication with a cartridge inlet 51 when the cartridge 50 is positioned in the cartridge receiving portion 10. The pumping mechanism 20 has a pump outlet 21 and the selection mechanism 30 is configured to align the pressurized air inlet 11 of the selected cartridge receiving portion 10 with the pump outlet 21. The device 1 includes a controller (not shown) configured to control the operation of the selection mechanism 30 and the pumping mechanism 20. The controller determines that the selected cartridge receiving portion is in its designated position and the fluid communication has been established, and accordingly starts the operation of the pumping mechanism 20. The pumping mechanism 20 generates the pressurized air and delivers the same to the cartridge 50 via the fluid communication between the pumping mechanism 20 and the cartridge receiving portion 10.

The cartridge 50 has a reservoir 52 containing an aromatic liquid 53 that is desired to be diffused and be delivered to the atmosphere around the liquid diffusing device 1. The cartridge 50 has a venturi device 54 configured to diffuse the liquid contained in the reservoir 52 by the action of the pressurized air and generate an aerosol flow. The aerosol flow is discharged through a cartridge outlet 55.

The device 1 further includes a blower 40 configured to generate a blown air that flows through a central air conduit 41 towards the cartridge outlet 55 and then is discharged in the blowing direction D. The controller is configured to operate the pumping mechanism 20 and the blower 40 in coordination such that the pressurized air from the pumping mechanism 20 carries therealong the aerosol flow through the cartridge outlet 55, and at the same time the blown air from the blower 40 is discharged through a blown air outlet 42 of the central air conduit 41. The blown air interacts with the aerosol flow and directs the same into the blowing direction D. The blowing direction D defines the intended direction of the aromatic spray into the atmosphere surrounding the device 1.

The controller is configured to control the selection mechanism 30 to select a next one of the cartridge receiving portion, for example 10′, and establish the fluid communication thereof with the pumping mechanism 20, and in the similar manner as described above, the aerosol flow from the corresponding cartridge is discharged into the atmosphere.

As can be seen in FIGS. 1A and 1B, the device includes an outer body 2, an electronic panel 3, and a power supply 4, which have been removed in FIGS. 1C to 1E for the purposes of illustration. The electronic panel has display unit to display the status of operation of the device 1, input means for a user to provide inputs for the controller to control the operation of the device 1, sensors to sense various atmospheric conditions that can be used by the controller to control the operation of the device 1. For instance, the device can have a reader module (not shown) that can read a corresponding code positioned on the cartridge to receive instructions for the controller for operating the selection mechanism, pumping mechanism, and the blower accordingly with respect to that cartridge. In some examples, the device can be configured to receive instructions from and transmit data to a user device remotely through a wireless connection. In some examples, the device can include a controller readable memory and the instructions can be stored on the memory. The instructions can include predetermined patterns of operation of the selection mechanism, pumping mechanism, and the blower. For instance, the instructions can define a duration of operation for each one of the cartridges, the pressure of the pumping mechanism for each one of the cartridges, the speed and duration of operation of the blower for each one of the cartridges, etc.

The liquid diffusing device 1 further includes fluid combiners 45, 45′, and 45″ associated with the cartridge receiving portions 10, 10′, and 10″ respectively. The fluid combiner 45 has a first combiner inlet 45A configured to be in fluid communication with the blower 40 via the central air conduit 41 when the corresponding cartridge receiving portion 10 is in fluid communication with the pumping mechanism 20. The fluid combiner 45 has a second combiner inlet 45B in fluid communication with the cartridge outlet 55 to receive the aerosol flow therefrom. The fluid combiner receives the blown air from the blower through the first combiner inlet 45A and the aerosol flow from the second combiner inlet 45B. The fluid combiner 45 includes a combiner body 46 defining a fluid channel 46A extending between the first combiner inlet 45A and a combiner outlet 45C. The fluid channel 46A constitutes a fluid path for the blown air and the aerosol flow, and facilitates interaction between the two. The blown air directs the aerosol flow in the blowing direction D and discharges it through the combiner outlet 45C.

When the selection mechanism 30 selects a next one of the cartridge receiving portion, for example 10′, and establish the fluid communication thereof with the pumping mechanism 20, the corresponding fluid combiner 45′ aligns with the blown air, and in the similar manner as described above, the aerosol flow from the corresponding cartridge is discharged into the atmosphere.

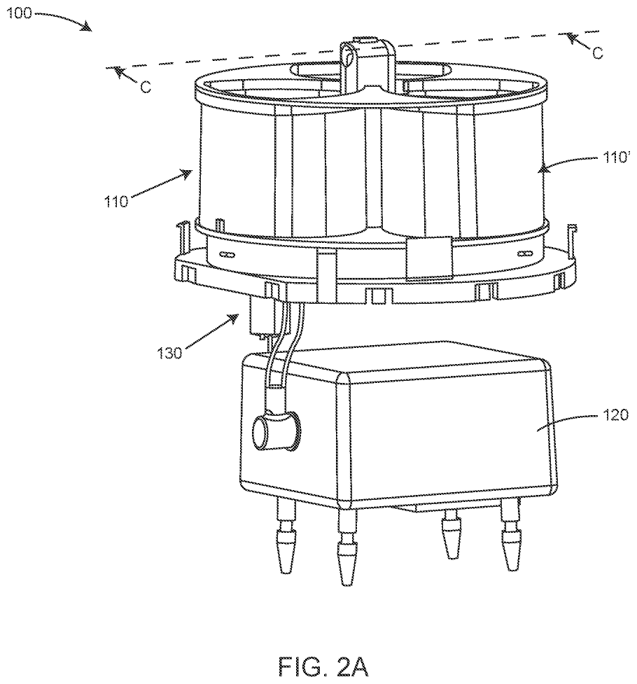

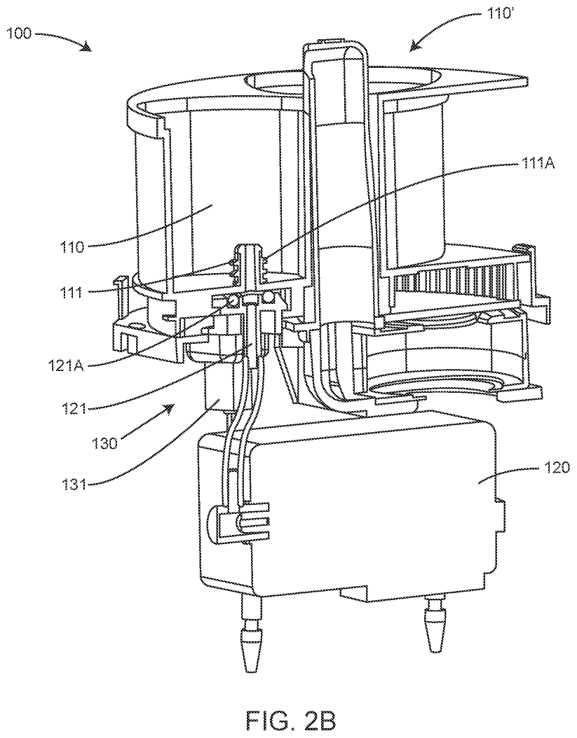

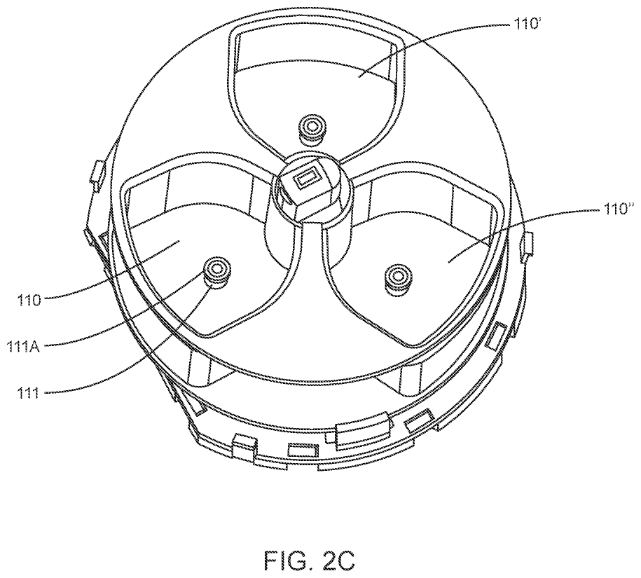

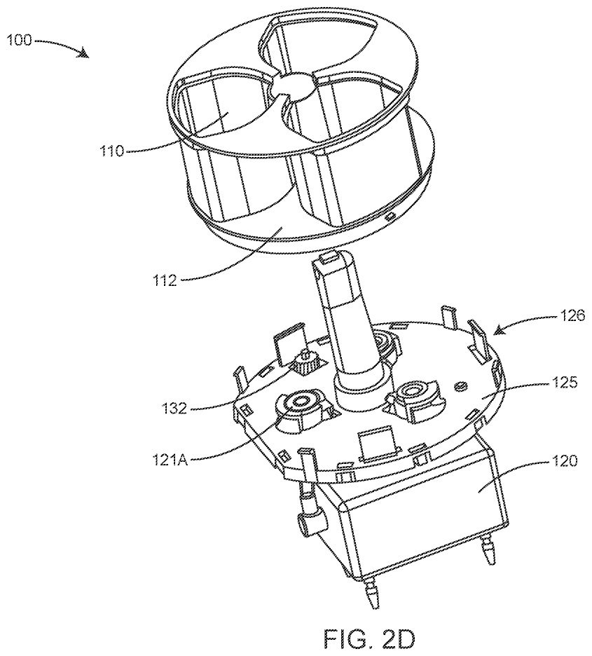

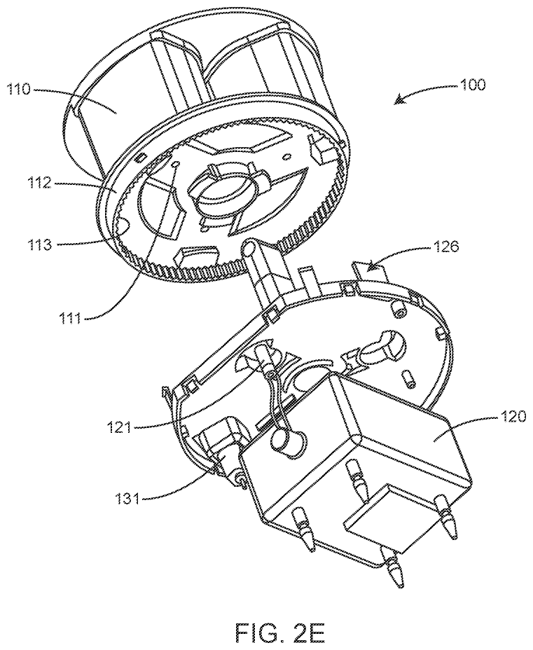

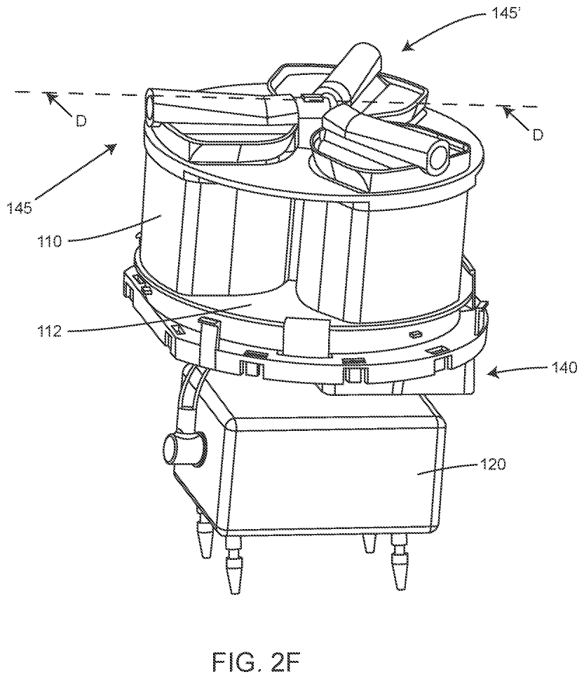

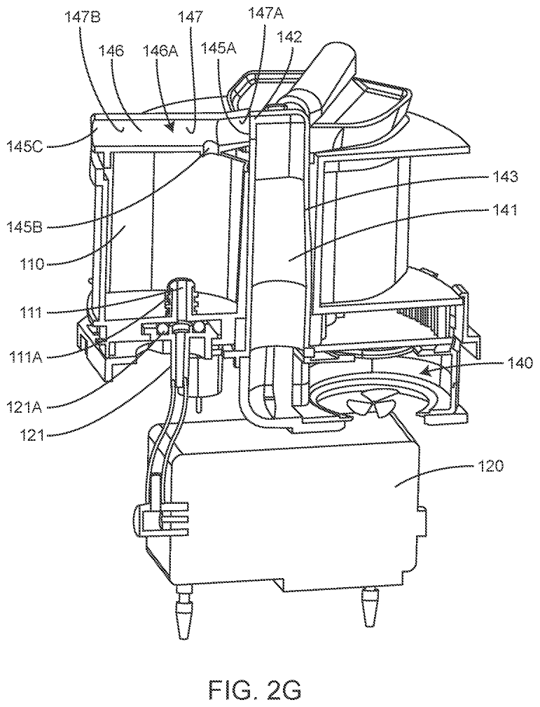

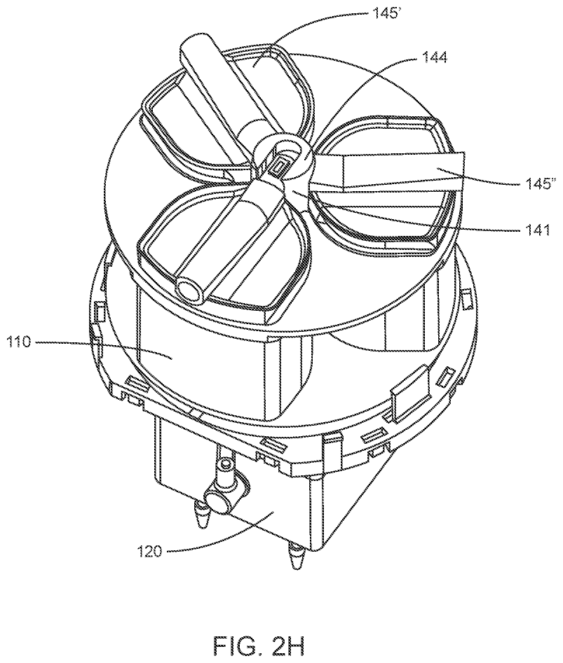

Reference is now made to FIGS. 2A to 2H to describe a liquid diffusing device 100 in accordance with another example of the presently disclosed matter. It is to be understood herein that the liquid diffusing device 100 has at least some of the components similar to those of the liquid diffusing device 1, and some of them have been excluded for the purposes of simplicity in illustration and description.

The liquid diffusing device has three cartridge receiving portions 110, 110′, and 110″, each configured to receive a corresponding removable cartridge similar to the cartridge 50 as detailed above. It is to be understood herein that for the purposes of the present description, the cartridge receiving portion is considered to be whole such portion of the liquid diffusing device that engages and/or contributes in holding the cartridge when the cartridge is introduced and positioned within the liquid diffusing device. In the illustrated example, three cartridge receiving portions have been shown, however, in other examples, the liquid diffusing device can have lesser or more number of cartridge receiving portions. The description below details one of the three cartridge receiving portions, i.e., 110 and it is to be understood that the description applies to the other cartridge receiving portions 110′ and 110″ as well.

The cartridge receiving portion 110 has pressurized air inlet 111 associated therewith and configured to be in fluid communication with a cartridge inlet (for example, the cartridge inlet 51) when the cartridge is positioned in the cartridge receiving portion 110. The pressurized air inlet 111 comprises an inlet sealing member 111A configured to seal the connection between the pressurized air inlet 111 and the cartridge inlet when the cartridge is positioned in the cartridge receiving portion 110. The liquid diffusing device 100 further includes a pumping mechanism 120 configured to generate pressurized air and pump the same into the cartridge to diffuse an aromatic liquid contained therein to generate an aerosol flow therefrom.

A selection mechanism 130 of the liquid diffusing device 100 is configured to selectively establish a fluid communication between a selected one of the cartridge receiving portions, which in the illustrated example has been shown to be cartridge receiving portion 110, and the pumping mechanism 120. In the illustrated example, the selection mechanism 130 is configured to selectively align the pressurized air inlet 111 of the selected cartridge receiving portion 110 with a pump outlet 121 to establish said fluid communication. In other examples, the pumping mechanism can be in fluid communication with each of the cartridge receiving portions and the selection mechanism can be configured to selectively open and close those fluid communications.

The pump outlet 121 has an outlet sealing member 121A that seals the fluid communication between the pump outlet 121 and the pressurized air inlet 111 when the pressurized air inlet 111 is aligned with the pump outlet 121. The outlet sealing member 121A seals the connection between the pump outlet 121 and the pressurized air inlet 111 and simultaneously allows the pressurized air inlet 111 to slide over the pump outlet 121 when the selection mechanism 130 moves the cartridge receiving portion 110, as described in detail herein below.

The cartridge receiving portions 110, 110′, and 110″ constitute a part of a movable assembly, which in the illustrated example is a cartridge receiving tray 112 configured in the form of a carousel-like arrangement and is configured to rotate about a longitudinal axis LA. The pumping mechanism 120 constitutes a part of a stationary assembly, which in the illustrated example includes a base member 125 and the pumping mechanism 120. The cartridge receiving tray 112 is movably mounted to the base member 125 and the selection mechanism 130 is configured to move the cartridge receiving tray 112 with respect to the base member 125. In the illustrated example, the selection mechanism comprises a motor 131 and a gear arrangement 132 positioned at the base member 125 and teeth 113 corresponding to the gear arrangement 132 positioned at the cartridge receiving tray 112. The gear arrangement 132 and the teeth 113 constitute a part of a mounting mechanism 126 configured to movably mount the cartridge receiving tray 112 to the base member 125. The mounting mechanism 126, in addition to the gear arrangement 132 and the teeth 113, also included holding elements 127 configured to firmly hold the cartridge receiving tray 112 in position during movement thereof. The selection mechanism 130 is configured to rotate the cartridge receiving tray 112 about a rotational axis, which in this case is the longitudinal axis LA, with respect to the base member 125. The selection mechanism 130 is configured to selectively align each (one at a time) of the cartridge receiving portions such that the corresponding pressurized air inlet is aligned with and in fluid communication with the pump outlet 121, which in the illustrated example is formed in the base member 125. Any such cartridge receiving portion whose associated pressurized air inlet is in fluid communication with the pumping mechanism is to be considered as the selected cartridge receiving portion at that time for the purpose of understanding of the present description.

The liquid diffusing device 100 has a controller (not shown) that is configured to control the operation of the selection mechanism 130 for selecting the cartridge receiving portions and aligning the selected cartridge receiving portion, 110 in the illustrated example, such the corresponding pressurized air inlet 111 is aligned with and in fluid communication with the pump outlet 121. The controller controls the operation of the motor 131 to rotate the cartridge receiving tray 112 with respect to the base member 125 to cause the alignment and establishment of the fluid communication. The controller can be configured to operate according to the instructions fed to the controller in any of the manners and to follow any of the predetermined patterns as described above with regard to the liquid diffusing device 1.

The controller is configured to determine whether a cartridge receiving portion has a cartridge or not and can be configured to operate the pumping mechanism only if the selected cartridge receiving portion includes a cartridge. The controller is configured to control the operation of the pumping mechanism 120 in coordination with that of the selection mechanism 130. For instance, the controller switches on the pumping mechanism 120 only after the selected cartridge receiving portion 110 is in fluid communication with the pumping mechanism 120. In other words, only after having controlled the selection mechanism 130 to select one of the cartridge receiving portions and establish a fluid communication thereof with the pumping mechanism 120 does the controller switches on the pumping mechanism 120 to pump the pressurized air into the corresponding cartridge. Once a next cartridge receiving portion is to be selected, the controller switches off the pumping mechanism 120 prior to controlling the selection mechanism 130 to move the cartridge receiving tray 112. In other words, the controller switches off the pumping mechanism 120 prior to terminating the fluid communication between the previously selected cartridge receiving portion and the pumping mechanism 120.

In some examples, such as one illustrated in FIGS. 2F to 2H, the liquid diffusing device 100 further includes a blower 140, which in the illustrated example constitutes a part of the stationary assembly, and is configured to generate a blown air in a blowing direction D. The cartridge receiving portions 110, 110′ and 110″ are so aligned that when the selected cartridge receiving portion is in fluid communication with the pumping mechanism 120, a cartridge outlet of the corresponding cartridge is so positioned that the aerosol flow being discharged therefrom is aligned with the blown air generated by the blower 140. Thus, the device 100 so operates that the pumping mechanism 120 pumps the pressurized air into the cartridge causing the liquid contained therein to be diffused into the aerosol flow. The aerosol is then discharged from the cartridge outlet which is aligned with the blown air, that interacts with and directs the aerosol flow in the blowing direction into the atmosphere around the device 100.

The controller is configured to operate the blower 140 in coordination with the selection mechanism 130. For instance, the controller switches on the blower 140 only after the selected cartridge receiving portion 110 is aligned with the blown air. In other words, only after having controlled the selection mechanism 130 to select one of the cartridge receiving portions and align the same such that the corresponding cartridge outlet will be aligned with the blown air, does the controller switches on the blower 140 to direct the aerosol flow in the blowing direction. Once a next cartridge receiving portion is to be selected, the controller switches off the blower 140 prior to controlling the selection mechanism 130 to move the cartridge receiving tray 112 thereby de-aligning the cartridge receiving portion from the blown air.

In fact, the controller is configured to control the operation of the pumping mechanism 120, the selection mechanism 130, and the blower 140 in coordination with each other, i.e., to follow a sequence. First of all, the selection mechanism 130 is controlled to select a cartridge receiving portion, establish the fluid communication thereof with the pumping mechanism 120, and simultaneously position the cartridge outlet so as to be aligned with the blown air. Then, the blower is switched on. After that, the pumping mechanism 120 is switched on in order to pump the pressurized air and generate the aerosol flow. The blower is switched on prior to the pumping mechanism so that the blown air is already generated to blow the aerosol flow and prevent any wastage of the aerosol. Once a next cartridge receiving portion is to be selected, first of all the pumping mechanism 120 is switched off to stop the generation of the aerosol flow. Then, the blower is switched off. The blower 140 is switched off after the pumping mechanism 120 so that the blown air blows away the already generated aerosol and little or no aerosol is wasted. After that the selection mechanism 130 is controlled to move the cartridge receiving tray 112 to select the next one of the cartridge receiving portions.

As can be seen in FIGS. 2F and 2G, the liquid diffusing device 100 further has fluid combiners 145, 145′, and 145″ associated respectively with the cartridge receiving portions 110, 110′, and 110″. Although in the illustrated example, a fluid combiner associated with each of the cartridge receiving portions is shown, it is to be understood herein that in some examples, there can be only one fluid combiner constituting a part of the stationary assembly and the selected cartridge receiving portion can be aligned with that one fluid combiner in the manner similar as the cartridge receiving portion is aligned with the pump outlet. The description below details one of the three fluid combiners, i.e., 145 and it is to be understood that the description applies to the other fluid combiners as well.

The fluid combiner 145 has a combiner body 146 defining a fluid channel 146A therewithin. The combiner 145 further has a first combiner inlet 145A formed in the combiner body 146, and configured to be in fluid communication with the blower 140 to receive the blown air therefrom into the fluid channel 146A, a second combiner inlet 146B formed in the combiner body 146, and configured to be in fluid communication with a cartridge outlet of a cartridge to receive the aerosol flow into the fluid channel 146A, and a combiner outlet 145C in fluid communication with the fluid channel 146A for discharging the aerosol flow having directed by the blown air in the blowing direction D.

In the illustrated example, the fluid combiner 145 is associated with the cartridge receiving portion 110 so as to have the second combiner inlet 145B in fluid communication with the cartridge outlet of the cartridge positioned in the cartridge receiving portion 110. When the cartridge receiving portion 110 is the selected one of the cartridge receiving portions, the first combiner inlet is aligned with the blown air to receive the blown air into the fluid channel 146A. The blown air interacts with the aerosol flow inside the fluid channel and directs the aerosol flow out of the combiner outlet 145C in the blowing direction. Thus, the fluid channel 146A constitutes a fluid path for the blown air from the first combiner inlet 145A to the combiner outlet 145C, such that the second combiner inlet 145B is positioned between the first combiner inlet 145A and the combiner outlet 145C. As can be seen best in FIG. 2G, the second combiner inlet 145B is closer to the first combiner inlet 145A than to the combiner outlet 145C. Thus prevents a direct access by fingers into the cartridge, or prevents the dust to enter into the cartridge. Apart from that, this positioning of the second combiner inlet 145B allows a longer distance to the aerosol flow to travel within the fluid channel 146A thereby allowing more distance and time for the big droplets in the aerosol to settle down in the fluid channel in flow back into the cartridge. For instance, the fluid channel 146A has a channel internal surface 147 facing an interior of the fluid channel 146A. The channel internal surface 147 has a first surface portion 147A extending between the first combiner inlet 145A and the second combiner inlet 145B, and a second surface portion 147B extending between the combiner outlet 145C and the second combiner inlet 145B. As can be seen in FIG. 2G, the first and second surface portions 147A and 147B are sloped down into the second combiner inlet 145B so as to allow the left over liquid from the aerosol flow that accumulates on the channel internal surface 147 to flow back into the cartridge. The combiner body 146 is configured to constitute at least a part of a cover for the cartridge when the cartridge is positioned in the corresponding cartridge receiving portion 110.

The liquid diffusing device 100 further includes a central air conduit 141 in fluid communication with the blower 140 and having a blown air outlet 142 configured to discharge therefrom the blown air. The central air conduit 141 has a conduit body 143 extending along the longitudinal axis LA constitutes a part of the stationary assembly. The central air conduit 141 extends through the cartridge receiving tray 112 and the cartridge receiving portions 110, 110′, and 110″ are configured to rotate around the central air conduit 141.

The first combiner inlet 145A of the fluid combiner 145 corresponding to the selected cartridge receiving portion 110 is aligned with and in fluid communication with the blown air outlet 142 of the central air conduit 141. At the same time, the conduit body 143 is so aligned with the first combiner inlets of the other two fluid combiners 145′ and 145″ so as to prevent access by fingers into those fluid combiners and corresponding cartridges. In some examples, such as the one illustrated in FIG. 2H, the liquid diffusing device 100 has a conduit cover 144 positioned on top of the conduit body 143 such that the conduit cover 144 prevents access into first combiner inlets of the fluid combiners corresponding to the non-selected cartridges.

It is to be understood herein that in some examples, the liquid diffusing device 100 may not include the blower and in such examples, the central air conduit 141 may also not be present and the liquid diffusing device can have an empty central path instead or that central path can accommodate any other components of the liquid diffusing device. Also, in such examples, the liquid diffusing device may not include the fluid combiners.

In some examples, the liquid diffusing device can include at least one cartridge receiving portion and a controller to control the operation of the pumping mechanism and the blower in coordination with each other. For instance, upon receiving a signal to operate the device, the controller is configured to switch on the blower prior to switching on the pumping mechanism, and upon receiving a signal to turn off the device, the controller is configured to switch off the pumping mechanism prior to switching off the blower.

In some examples, the liquid diffusing device can include the fluid combiner with only one cartridge receiving portion and without any selection mechanism. For instance, such a liquid diffusing device can have a cartridge (removable or fixed) to generate an aerosol flow, a blower to direct the aerosol flow, and a fluid combiner similar to the one described above to facilitate the above described interaction between the aerosol flow and blown air from the blower.

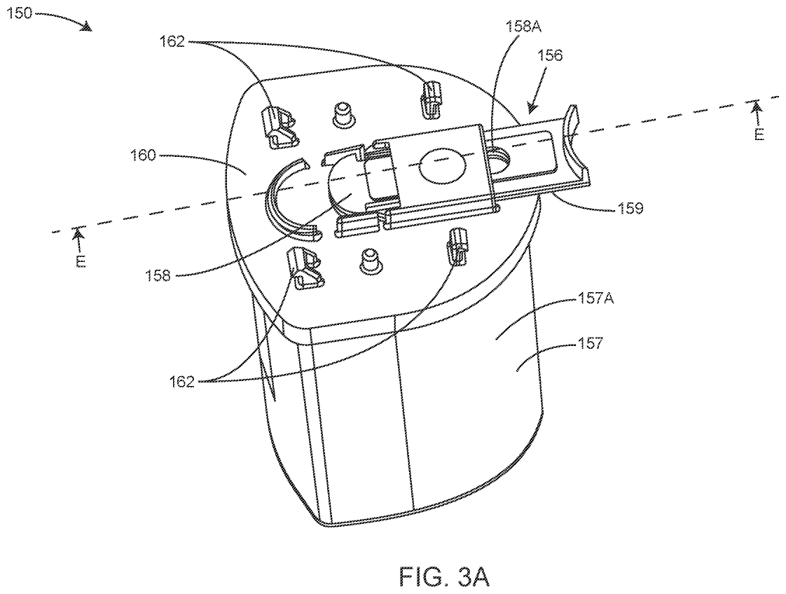

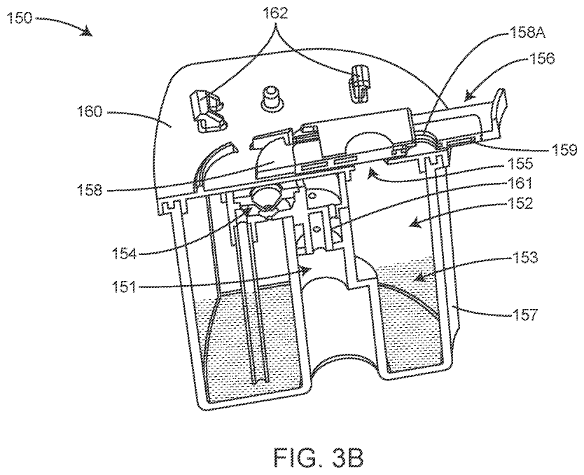

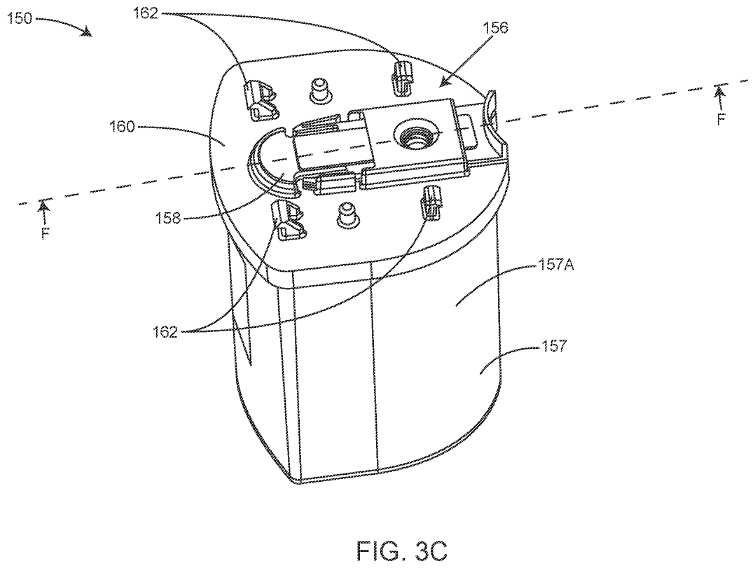

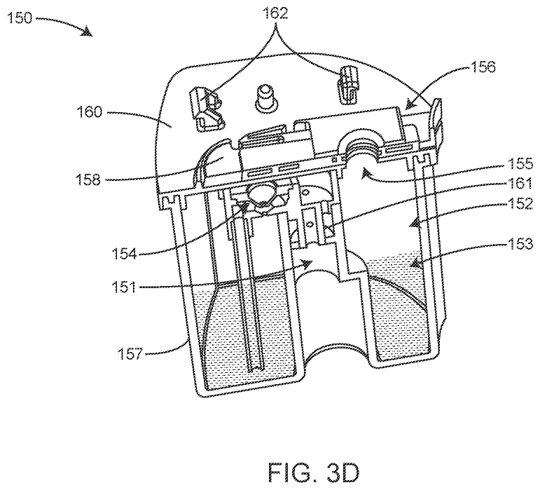

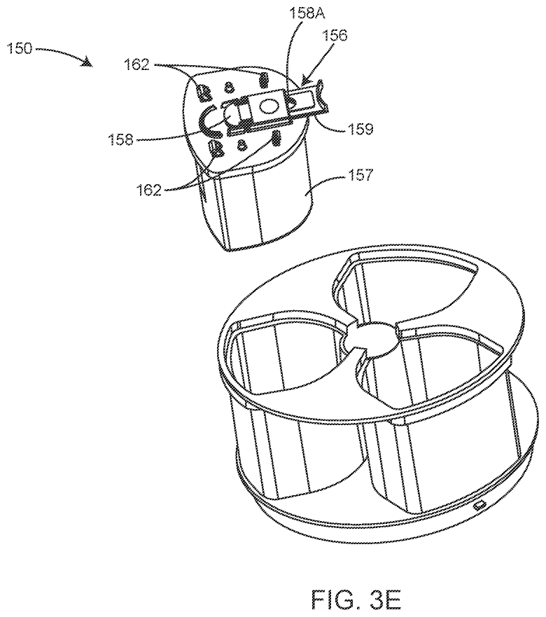

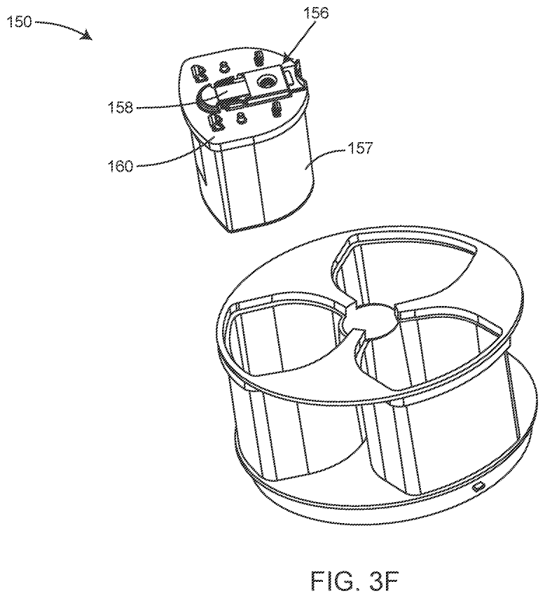

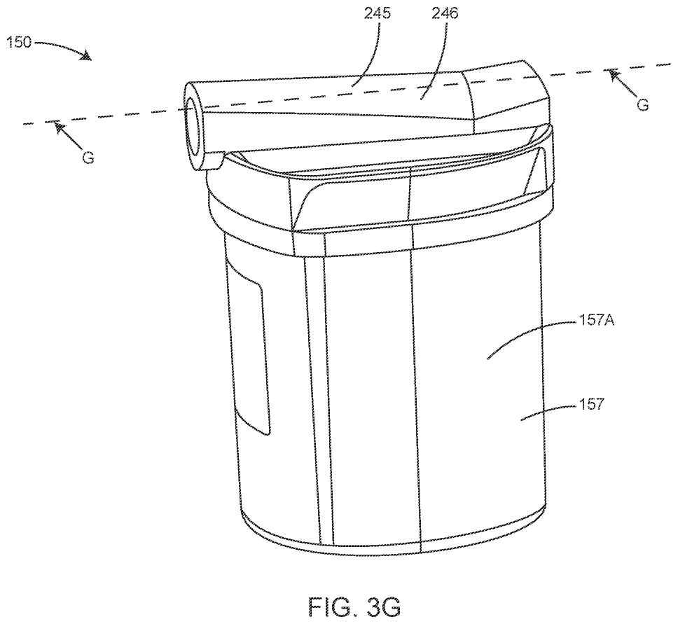

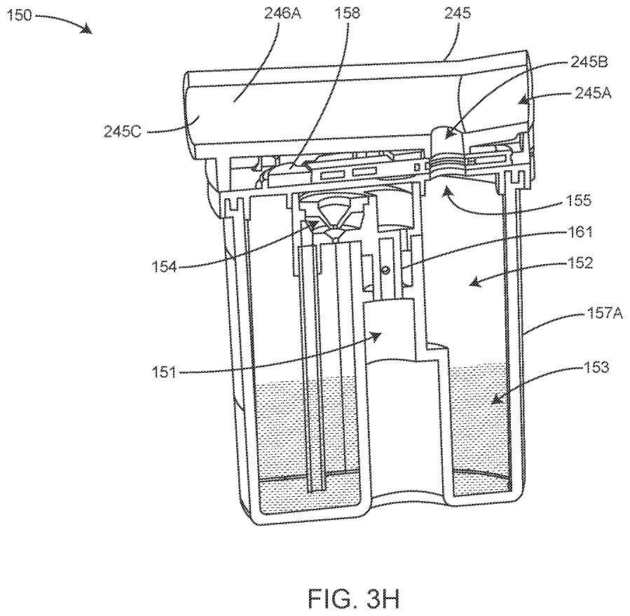

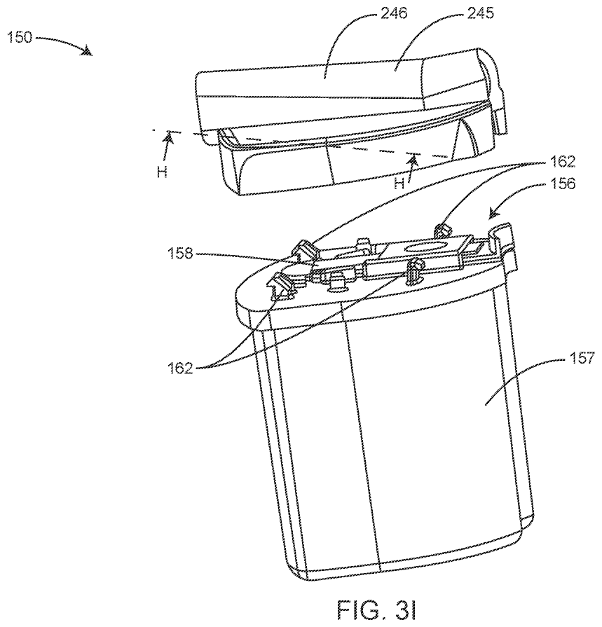

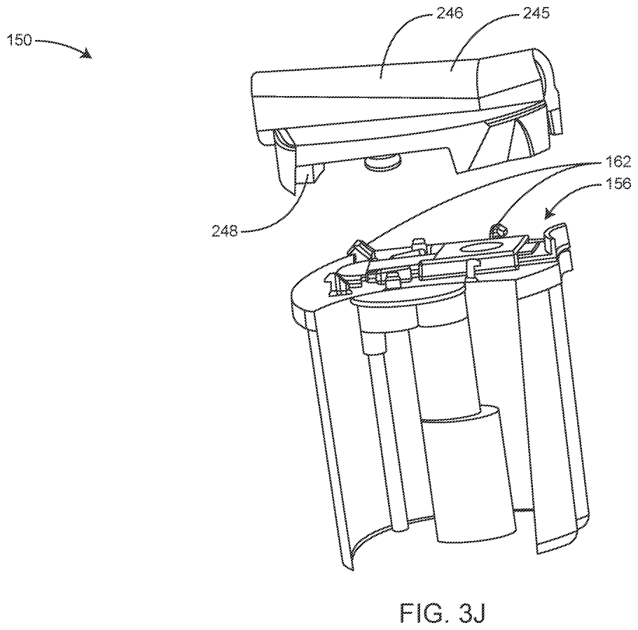

Reference is now made to FIGS. 3A to 3J to describe a cartridge 150 in accordance with an example of the presently disclosed matter. It is to be understood herein that the cartridge 150 has at least some of the components similar to those of the cartridge 50.

The cartridge 150 can be used with a liquid diffusing device, for example the liquid diffusing devices 1 and/or 100, for delivering aromatic spray into an environment such as a room or an office space. The cartridge 150 operates in a similar manner as the cartridge 50 described above. For instance, the cartridge 150 receives pressurized air through a cartridge inlet 151 and has a reservoir 152 containing an aromatic liquid 153 to be diffused into an aerosol flow by action of the pressurized air and a venturi device 154. The aerosol flow is then delivered through a cartridge outlet 155. In addition to the components described above, the cartridge 150 also includes a locking mechanism 156. The locking mechanism 156 is configured to displace between a closed state, as shown in FIGS. 3A and 3B, and an open state, as shown in FIGS. 3C and 3D. As can be seen in FIG. 3B, in the closed state of the locking mechanism 156, the cartridge outlet 155 is sealed, and as can be seen in FIG. 3D, at the open state of the locking mechanism 156, the cartridge outlet is open. The locking mechanism 156 in its closed state is configured to prevent the introduction of the cartridge in the liquid diffusing device, one example of which is described below.

The cartridge 150 has a cartridge body 157 and the locking mechanism 156 has an obstructing member 158 that protrudes from the cartridge body 157 in the closed state of the locking mechanism 156, as can be seen in FIGS. 3A, 3B, and 3E. For instance, as can be best seen in FIG. 3E, the obstructing member 158 has an obstructing portion 159 that protrudes further from a contour defined by the cartridge body 157 and would engage with a rim of the cartridge receiving portion of the liquid diffusing device, for example the rim R of the cartridge receiving portion 110 of the liquid diffusing device 100. It is to be understood herein that the liquid diffusing device can be any other liquid diffusing device having dimensions of the cartridge receiving portions corresponding to those of the cartridge with its locking mechanism in open state, and with the obstructing member extending beyond those dimensions in the closed state to engage any portion of the liquid diffing device for preventing the introduction of the cartridge. In the illustrated examples, the cartridge body 157 has a body side wall 157A and the obstructing portion 159 protrudes radially from the body side wall 157A in the closed state of the locking mechanism 156. The body side wall 157A has a shape and size corresponding the shape and size of the cartridge receiving portion 110 and the obstructing portion 159 extending radially from the body side wall 157A engages the rim R of the cartridge receiving portion 110 thereby preventing the introduction of the cartridge into the cartridge receiving portion. As can be seen in FIG. 3F, in the open state of the locking mechanism 156, the obstructing member 158 is displaced radially inwardly and the obstructing portion 159 lies within the contour defined by the body side wall 157A. In such a state, the cartridge 150 can be inserted into the cartridge receiving portion 110.

In the illustrated example, the locking mechanism 156 constitutes a part of a cover 160 of the cartridge 150. The obstructing member 158 has an orifice 158A that corresponds in shape and size to the cartridge outlet 155 and in the open state of the locking mechanism 156, the orifice 158A aligns with the cartridge outlet 155 thereby opening the outlet for the aerosol flow to be discharged therefrom. In the locked state of the locking mechanism 156, the orifice 158A is not aligned with the cartridge outlet 155 and the obstructing member 158 seals the outlet. Thus, the locking mechanism 156 simultaneously allows introduction of the cartridge 150 into the corresponding cartridge receiving portion and opens the cartridge outlet 155 for use thereof. In some examples, the locking mechanism 156 can have a biasing member configured to bias the obstructing member 158 so as to keep the locking mechanism into its closed state, such that as soon as the cartridge 150 is removed from the cartridge receiving portion 110, the biasing member displaces the locking mechanism into its closed state thereby sealing the cartridge outlet. This prevents spillage of the liquid from the cartridge through the cartridge outlet 155. Further, the cartridge 150 includes a one-way valve, which in the illustrated example is a sleeve 161, at the cartridge inlet 151 configured to allow the pressurized air to enter the cartridge while simultaneously preventing the spillage of the liquid from the cartridge inlet 151.

In some examples, such as the one illustrated in FIGS. 3G to 3J, the cartridge 150 includes a fluid combiner 245. It is to be understood herein that the fluid combiner 245 has at least some of the components similar to, and operates in the similar manner as, those of the fluid combiners 45 and/or 145. For instance, the fluid combiner 245 has a combiner body 246 defining a fluid channel 246A therewithin. The combiner 245 further has a first combiner inlet 245A formed in the combiner body 246, and configured to be in fluid communication with a blower of the liquid diffusing device to receive blown air therefrom into the fluid channel 246A, a second combiner inlet 245B formed in the combiner body 246, and configured to be in fluid communication with the cartridge outlet 155 to receive the aerosol flow into the fluid channel 246A, and a combiner outlet 245C in fluid communication with the fluid channel 246A for discharging the aerosol flow having directed by the blown air.

As can be seen in FIGS. 3G to 3J, the second combiner inlet 245B is aligned with the cartridge outlet 155 and is in fluid communication with the same through the orifice 158A when the cartridge outlet 155 is opened by the locking mechanism 156. The first combiner inlet 245A is configured to be in fluid communication with the blower of the liquid diffusing device as described above with reference to the fluid combiners 45 and 145. It is to be understood herein that all or some of the features about the fluid channel, the internal surface, the combiner inlets, the combiner outlet, and/or the operation of the combiner as described above for the fluid combiner 145 apply to the fluid combiner 245 as well. In addition to that, the fluid combiner 245 comprises combiner connection elements 248 configured to be connected to the cartridge 150. The cartridge cover 160 has corresponding cartridge connection elements 162 for connecting to the combiner connection elements 248. The combiner connection elements 248 and the cartridge connection elements 162 constitute a quick-fitting connection arrangement such as a snap connection. In other examples, the connection arrangement can be any other quick-connection arrangement know to a skilled person.

As can be seen in FIGS. 3H to 3J, the locking mechanism 156 is positioned between the fluid combiner 245 and the cartridge 150 when the fluid combiner 245 is connected to the cartridge 150. In some examples, the cartridge 150 can include the fluid combiner 245 without the locking mechanism and can operate in the same manner as described above. For instance, such a cartridge can be used with a liquid diffusing device including a blower to direct the aerosol flow and can include a fluid combiner similar to the one described above to facilitate the above-described interaction between the aerosol flow and blown air from the blower.

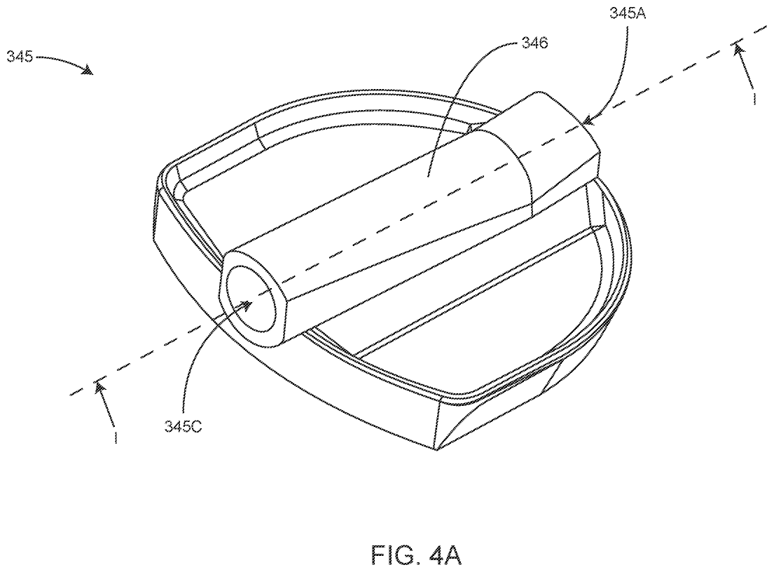

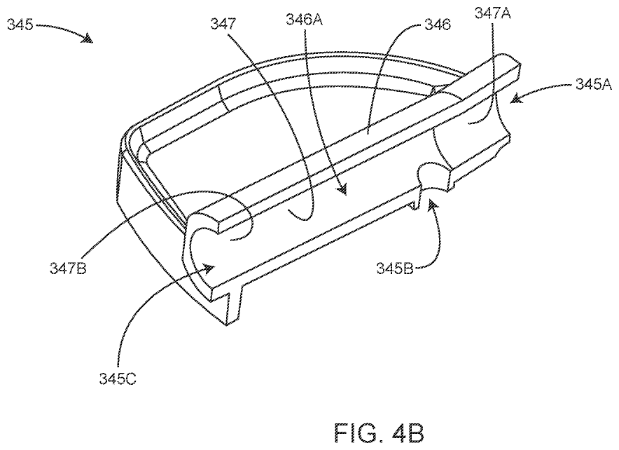

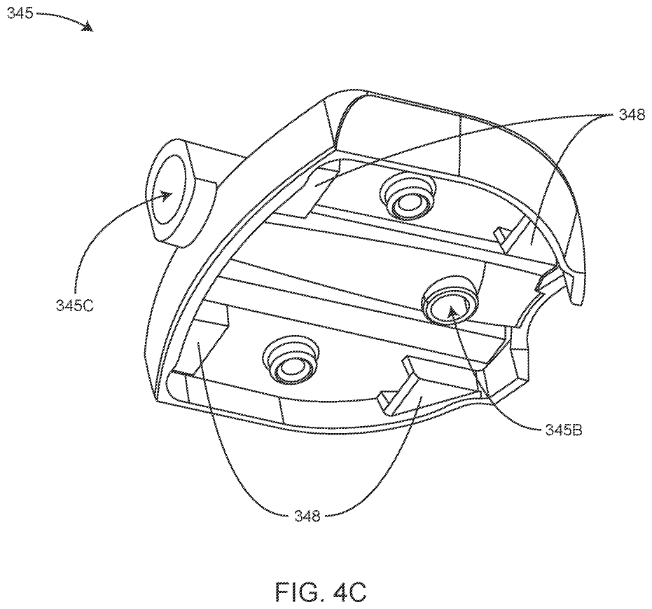

Reference is now made to FIGS. 4A to 4C to describe a fluid combiner 345 in accordance with an example of the presently disclosed matter. It is to be understood herein that the fluid combiner 345 has at least some of the components similar to, and operates in the similar manner as, those of the fluid combiners 45, 145, and/or 245 as described above. The fluid combiner 345 can constitute a part of a liquid diffusing device such as, but not limited to, the liquid diffusing device 1 and/or 100 or a cartridge such as, but not limited to, the cartridge 50 and/or 150. Alternatively, the fluid combiner 345 can be a stand-alone element to be used with any liquid diffusing device having a blower to direct a generated aerosol flow or with any cartridge that is to be used with such a liquid diffusing device.

For instance, the fluid combiner 345 has a combiner body 346 defining a fluid channel 346A therewithin. The combiner 345 further has a first combiner inlet 345A formed in the combiner body 346, and configured to be in fluid communication with a blower of the liquid diffusing device to receive blown air therefrom into the fluid channel 346A, a second combiner inlet 345B formed in the combiner body 346, and configured to be in fluid communication with a cartridge outlet of the cartridge to receive the aerosol flow into the fluid channel 346A, and a combiner outlet 345C in fluid communication with the fluid channel 346A for discharging the aerosol flow having directed by the blown air.

It is to be understood herein that all or some of the features about the fluid channel, the internal surface, the combiner inlets, the combiner outlet, and/or the operation of the combiner as described above for the fluid combiner 145 apply to the fluid combiner 245 as well. In addition to that, the fluid combiner 245 comprises combiner connection elements 248 configured to be connected to the cartridge 150.

During operation of the fluid combiner 345, the blown air interacts with the aerosol flow inside the fluid channel 346A and directs the aerosol flow out of the combiner outlet 345C in the blowing direction, as described above with reference to the fluid combiner 145 above. Thus, the fluid channel 346A constitutes a fluid path for the blown air from the first combiner inlet 345A to the combiner outlet 345C, such that the second combiner inlet 345B is positioned between the first combiner inlet 345A and the combiner outlet 345C. As can be seen best in FIG. 4B, the second combiner inlet 345B is closer to the first combiner inlet 345A than to the combiner outlet 345C. This prevents a direct access by fingers into the cartridge or prevents the dust to enter into the cartridge. Apart from that, this positioning of the second combiner inlet 345B allows a longer distance to the aerosol flow to travel within the fluid channel 346A thereby allowing more distance and time for the big droplets in the aerosol flow to settle down in the fluid channel in flow back into the cartridge. For instance, the fluid channel 346A has a channel internal surface 347 facing an interior of the fluid channel 346A. The channel internal surface 347 has a first surface portion 347A extending between the first combiner inlet 345A and the second combiner inlet 345B, and a second surface portion 347B extending between the combiner outlet 345C and the second combiner inlet 345B. As can be seen in FIG. 4B, the first and second surface portions 347A and 347B are sloped down into the second combiner inlet 345C so as to allow the left-over liquid from the aerosol flow that accumulates on the channel internal surface 347 to flow back into the cartridge.

The combiner body 346 is configured to constitute at least a part of a cover for the cartridge and for that, the fluid combiner 345 comprises combiner connection elements 348 configured to be connected to the corresponding connection elements of the cartridge. In some examples the combiner can include connection elements connectable to the corresponding connection elements of the liquid diffusing device instead of or in addition to the connection elements 248.

1-61. (canceled)

62. A liquid diffusing device comprising:
at least two cartridge receiving portions, each of the at least two cartridge receiving portions is configured for removably receiving a cartridge configured to contain a liquid to be diffused, and having a respective pressurized air inlet associated therewith and configured to be in fluid communication with a cartridge inlet when the cartridge is positioned in the cartridge receiving portion;
a pumping mechanism configured to generate pressurized air;
a selection mechanism configured to selectively establish a fluid communication between the pumping mechanism and the pressurized air inlet of one of the cartridge receiving portions for the pumping mechanism to pump the pressurized air into the respective cartridge for diffusing the liquid contained therein and generating an aerosol flow; and
a controller configured for controlling the operation of the selection mechanism to select the cartridge receiving portions to establish the fluid communication between the selected cartridge receiving portion with the pumping mechanism.

63. The liquid diffusing device of claim 62, wherein the pumping mechanism comprises a pump outlet, and the selection mechanism is configured to selectively align the pressurized air inlet of said one of the cartridge receiving portions with said pump outlet to establish said fluid communication therebetween.

64. The liquid diffusing device of claim 62, wherein said at least two cartridge receiving portions constitute at least a part of a movable assembly, and said pumping mechanism constitutes at least a part of a stationary assembly.

65. The liquid diffusing device of claim 64, wherein said selection mechanism is configured to move the movable assembly with respect to the stationary assembly.

66. The liquid diffusing device of claim 62, wherein said controller is configured to determine whether a cartridge receiving portion has a cartridge or not.

67. The liquid diffusing device of claim 62, wherein said controller is configured to control the operation of the pumping mechanism in coordination with that of the selection mechanism.

68. The liquid diffusing device of claim 62, wherein the controller is configured to provide said coordination by switching off the pumping mechanism prior to said selection mechanism terminating the fluid communication between said at least one of the cartridge receiving portions and the pumping mechanism.

69. The liquid diffusing device of claim 62, wherein the controller is configured for controlling the operation of the pumping mechanism according to a predetermined pattern.

70. The liquid diffusing device of claim 62, further comprising a blower configured to generate a blown air directed towards a blowing direction.

71. The liquid diffusing device of claim 70, wherein the selection mechanism is configured to selectively align said one of the cartridge receiving portions with the blown air and thereby causing the aerosol flow to be directed by the blown air towards the blowing direction.

72. The liquid diffusing device of claim 70, wherein said controller is configured to control the operation of the pumping mechanism in coordination with that of the selection mechanism; and wherein said controller is configured to control the operation of the blower in coordination with that of the pumping mechanism.

73. The liquid diffusing device of claim 70, further comprising a fluid combiner, said fluid combiner comprising:
a combiner body with a fluid channel;
a first combiner inlet formed in the combiner body, and configured to be in fluid communication with the blower to receive the blown air therefrom into the fluid channel;
a second combiner inlet formed in the combiner body, and configured to be in fluid communication with a cartridge outlet of a cartridge to receive the aerosol flow into the fluid channel; and
a combiner outlet in fluid communication with the fluid channel for discharging the aerosol flow having directed by the blown air in the blowing direction.

74. The liquid diffusing device of claim 73, wherein the fluid channel comprises a channel internal surface facing an interior of the fluid channel and having a first surface portion extending between the first combiner inlet and the second combiner inlet, and a second surface portion extending between the combiner outlet and the second combiner inlet, wherein said first and second surface portions are sloped down into the second combiner inlet.

75. The liquid diffusing device of claim 73, wherein the fluid combiner is associated with at least one of the cartridge receiving portions, wherein the selection mechanism is configured to align said first combiner inlet with the blown air simultaneously with establishing the fluid communication between the pumping mechanism and said one of the cartridge receiving portions.

76. The liquid diffusing device of claim 73, wherein said fluid combiner constitutes at least a part of a cover for the cartridge at least when the cartridge is received in the corresponding cartridge receiving portion.

77. The liquid diffusing device of claim 73, wherein said selection mechanism is configured to move the movable assembly with respect to the stationary assembly; wherein said selection mechanism comprises a motor configured to rotate the movable assembly about a rotational axis with respect to the stationary assembly; wherein the liquid diffusing device comprises a central air conduit constituting a part of the stationary assembly; and wherein the central air conduit is in fluid communication with the blower and has a blown air outlet configured to discharge said blown air.

78. The liquid diffusing device of claim 77, wherein the liquid diffusing device comprises at least two fluid combiners, each associated with one the cartridge receiving portions, wherein the selection mechanism is configured to align said first combiner inlet of one of the fluid combiners with the blown air outlet simultaneously with establishing the fluid communication between the pumping mechanism and corresponding one of the cartridge receiving portions.

79. A liquid diffusing device comprising:
at least one cartridge receiving portion configured for removably receiving a cartridge configured to contain a liquid to be diffused;
a pumping mechanism configured to generate pressurized air, and to be in fluid communication with the cartridge for pumping the pressurized air into the cartridge for diffusing the liquid contained therein and generating an aerosol flow;
a blower configured to generate a blown air in a blowing direction, said blown air being configured to direct said aerosol flow in the blowing direction away from the liquid diffusing device; and
a controller configured to control the operation of the pumping mechanism and the blower in a predetermined coordinated manner.

80. The liquid diffusing device of claim 79, wherein the controller is configured to provide said coordination by switching on the blower prior to switching on the pumping mechanism.

81. The liquid diffusing device of claim 79, wherein the controller is configured to provide said coordination by switching off the pumping mechanism prior to switching off the blower.
